(19) United States
(12) Patent Application Publication
(10) Pub. No.: US 2015/0306112 A1
(43) Pub. Date: Oct. 29, 2015

(54) Zhankuic acid A, a JAK2/3 tyrosine kinase inhibitor, and a potential therapeutic agent for hepatitis

(71) Applicant: National Cheng Kung University, Tainan (TW)

(72) Inventors: Tian-Shung WU, Tainan (TW); Chao-Liang WU, Tainan (TW); Ai-Li SHIAU, Tainan (TW); Yu-Fon CHEN, Tainan (TW)

(21) Appl. No.: 14/695,548

(22) Filed: Apr. 24, 2015

Related U.S. Application Data
(60) Provisional application No. 61/984,139, filed on Apr. 25, 2014.

(51) Int. Cl. A61K 31/575

(52) U.S. Cl. CPC A61K 31/575

(57) ABSTRACT

Zhankuic acid A (ZAA) could suppress phosphorylation of JAK2 and JAK3 and signaling of downstream molecules. Moreover, ZAA could inhibit the IFN-γ/STAT1/IRF-1 pathway in vivo and in vitro. Furthermore, data show that pre-treatment with ZAA could significantly ameliorate Con A-induced hepatitis in mice. The above results strongly suggest that ZAA treatment could block JAK2 and JAK3 activation, and may be a valuable therapeutic approach for the treatment of immune cell induced inflammation.

### FIELD OF THE INVENTION

The present invention is related to a novel medical use of zhankuic acid A (ZAA) in treating a subject having a Janus tyrosine kinases (JAK)-associated disorder, and in particular the treatment of immune cell induced inflammation including hepatitis.

### ABBREVIATIONS USED IN THIS INVENTION

JAK2, Janus tyrosine kinases 2; JAK3, Janus tyrosine kinases; ZAA, Zhankuic acid A; STAT, signal transducer and activator of transcription; IRF-1, interferon regulatory factor 1

### BACKGROUND OF THE INVENTION

Janus tyrosine kinases (JAKs) expressed in immune cells regulate the signaling of multiple cytokines that are important for various immune cell functions [1]. JAK/signal transducer and activator of transcription (STAT) mediate signaling by IFNs, many interleukins and growth factors on the cell surface of the nucleus [2, 3]. Binding of cytokine receptors to the catalytic FERM domain of JAKs could activate JAKs, which creates docking sites for the STAT family [4]. Phosphorylated STATs form a dimer and translocate to the nucleus, bind to DNA and regulate target gene expression. Constitutive activation of JAK2 is associated with inflammatory cytokine expression such as IL-6, IFN-γ and granulocyte-macrophage colony-stimulating factor (GM-CSF) [5-8], and was observed in myeloproliferative disorders [9, 10]. Furthermore, JAK3 activation not only involved the development and survival of T-cells but also Th cell differentiation [11, 12]. Recent studies also showed that IL-2-stimulated JAK3 activation plays an important role in the proliferation and differentiation of lymphocytes and augments the cytolytic activity of NK-cells [13, 14]. Therefore, as immune disorders and autoimmune diseases continue to present as unmet medical need in inflammation, JAK2 and JAK3 become novel targets to develop innovative therapies.

A previous study showed that Zhankuic acid A (ZAA) exhibited cytotoxic activity against P-388 murine leukaemia cells [15]. The methanol extracts derived from the fruiting body of Taiwanofungus camphoratus could inhibit STAT1 activation in the LPS/IFN-γ-activated microglia [16] and anti-proliferative activity in Jurkat cells [17]. However, only a few mechanistic studies related to ZAA regulation of the anti-inflammatory-related signaling pathway have been reported.

### SUMMARY OF THE INVENTION

A primary objective of the present invention is to provide a method of treating a subject having a JAK-associated disorder, which comprises administering to said subject in need of said treatment of Zhankuic acid A or a pharmaceutically acceptable salt thereof.

Preferably, the JAK-associated disorder is a myeloproliferative disorder. Preferably, the myeloproliferative disorder is polycythemia vera (PV), essential thrombocythemia (ET), myeloid metaplasia with myelofibrosis (MMM), chronic myelogenous leukemia (CML), chronic myelomonocytic leukemia (CMML), hypereosinophilic syndrome (HES), or systemic mast cell disease (SMCD).

Preferably, the JAK-associate disorder is an immune disorder caused by organ transplant rejection.

Preferably, the JAK-associate disorder is an autoimmune disease.

Preferably, the JAK-associate disorder is an immune cell induced inflammation.

Preferably, the JAK-associate disorder is hepatitis.

### BRIEF DESCRIPTION OF THE DRAWINGS

FIG. 1. Effects of ZAA on T-cell proliferation induced by multiple stimulators and cytokine signaling in vitro. (A) C57/BL6 splenocytes were treated with stimulators for 48 h, followed by incubation with ZAA at the indicated concentrations for 48 h, to evaluate cell proliferation by MTS assay. (B) Splenocytes were treated with ZAA, ZAA and Con A for the indicated times for 24 h to determine the supernatant IFN-γ by ELISA. (C) Splenocytes were cultured with ZAA for 3 h and then treated with IFN-γ or IL-6 for 30 min. After the incubation, cell lysates were assessed by immunoblot. (D) Jurkat cells were cultured without or with Con A for 24 h, followed by incubation with ZAA at the indicated concentrations for 24 h to detect the caspases by immunoblot (upper). In the same condition, cell proliferation was evaluated at 24, 48 and 72 h by MTS assay (lower). (E) Jurkat and C57/BL6 CD4⁺-T cells were cultured without or with Con A for 24 h, followed by incubation with ZAA at the indicated concentrations for 24 h to evaluate IRF-1 expression by immunoblot. Data are expressed as the mean±SEM (n=4). **P, 0.01; ***P, 0.001 vs. stimulators-treated groups. Similar results were obtained in at least three independent experiments.

FIG. 2. ZAA interacts with the hydrophobic binding pocket of JAK2 and JAK3. (A) The human JAK2 kinase (PDB code: 4BBE) is complexed with a selective inhibitor, 304. (B) Human JAK2 kinase is complexed with ZAA. (C) The binding pocket of ZAA in JAK2. (D) Interactions involved in the binding of ZAA to the amino acid residues of JAK2. (E) Closer view of the ZAA-binding pocket in JAK2. (F) Immunoblotting with JAK2 antibody was performed. (G) Human JAK3 kinase (PDB code: 4HVI) is complexed with a selective inhibitor, 19S. (H) Human JAK3 kinase is complexed with ZAA. (I) The binding pocket of ZAA in JAK3. (J) Interactions involved in the binding of ZAA to the amino acid residues of JAK3. (K) Closer view of the ZAA-binding pocket in JAK3. (L) Immunoblotting with JAK3 antibody was performed.

FIG. 3. ZAA inhibited the phosphorylation of JAK2 and JAK3 and their downstream signaling. (A) Jurkat and THP-1 cells were treated with ZAA for 3 h, followed by stimulation with IFN-γ, IL-6, IL-2 or GM-CSF for 30 min. Total cell lysates were examined for the indicated proteins by immunoblotting. (B) Jurkat cells were transfected with p-GAS-Luc plasmids. After 24 h, the cells were treated with or without ZAA for 1 h and then treated with IFN-γ for 24 h. Total cell lysates were harvested, and their luciferase activities were determined and normalised to the total protein. (C) Jurkat and THP-1 cells were pre-treated without or with ZAA for 3 h, followed by incubation with IFN-γ for 24 h. Total mRNA was prepared, and the mRNA levels of IRF-1 and SOCS-1 were detected by RT-PCR. GAPDH was used as the reference band. (D) HepG2 cells were cultured with ZAA or ruxolitinib for 48 h. Total cell lysates were examined for the indicated proteins by immunoblotting. β-actin was used as internal control. (E) Jurkat cells were treated with ZAA for 3 h, followed by stimulation with human IFN-α (10 ng/mL), IFN-β (20 ng/mL) or IFN-γ (20 ng/mL) for 30 min. Total cell lysates were examined for the indicated proteins by immunoblotting. All data are one of three independent experiments with similar results.

FIG. 4. ZAA protected mice against Con A-induced liver injury. C57BL/6 mice were intraperitoneally administered the vehicle or 20 mg/kg ZAA, 1 h prior to i.v. injection with Con A (15 mg/kg). (A) Representative microphotographs showing liver and spleen histopathologic changes with hematoxylin-and-eosin-staining (original magnification ×200). (B) The necrosis was analysed with a four-point score: 0, no; 1, 1-20% necrosis; 2, 20-40% necrosis; 3, >40% necrosis. Infiltration of leukocytes was graded on a four-point severity scale: 0, none; 1, little; 2, moderate; 3, mass. (C) Serum ALT and AST activities were measured at 8 h after Con A injection. (D) Serum levels of IFN-γ, IL-6 and IL-4 were measured at 8 h after Con A injection by ELISA. (E) Proteins from liver and spleen tissues of vehicle, ZAA and dexamethasone-treated mice were extracted and subjected to immunoblot. Values are shown as mean±S.E.M. from individual mice (n=10-13 mice/group). ***P<0.001,**P<0.01, *P<0.05 as compared with vehicle-treated mice. Similar results were obtained in at least three independent experiments.

FIG. 5. (A) Splenocytes from BALB/c mice, that had been treated with mitomycin C (25 μg/mL) for 1 h, were cocultured with splenocytes from C57/BL6, in the presence or absence of the indicated concentrations of ZAA for 72 h. Cell proliferation was measured by MTS assay. ***P<0.001, **P<0.01, *P<0.05 when compared with the control group. (B) and (C) Jurkat cells were cultured with ZAA for 3 h, then treated with various concentrations of IFN-γ for 30 min. (D) and (E) Raw264.7 cells were cultured with ZAA for 3 h, then treated with various concentrations of IL-6 for 30 min. Following incubation, proteins were extracted and assessed by immunoblot analysis. Similar results were obtained in at least three independent experiments.

FIG. 6. The protein structures of JAK1, JAK2, JAK3 and TYK2 were used for docking with ZAA. The PDB codes for JAK1, JAK2, JAK3 and TYK2 were 4KF6, 4BBE, 4HVI and 3LXP, respectively. The ZAA was found to bind to the substrate-binding sites on JAK2 and JAK3 rather than JAK1 and TYK2. The amino acid residues in the regions of substrate-binding sites are highly conserved in these four kinases.

FIG. 7. IFN-γ-activated STAT1 and induced IRF-1 protein expression in Jurkat, THP-1, Chang liver and HMEC-1 cells. Cells were cultured without or with ZAA in serum-free medium for 3 h, and then stimulated with IFN-γ (20 ng/mL). After various time periods, cells were harvested, and total protein extracts were prepared for imunoblot analysis using the indicated antibodies. Similar results were obtained in at least three independent experiments.

### DETAILED DESCRIPTION OF THE INVENTION

### Aims

Janus tyrosine kinases (JAK) activation is involved in the signaling of several cytokines that are important for various immune cell functions, and are very intriguing therapeutic targets for inflammatory-related diseases. Recent studies have shown that zhankuic acid A (ZAA) is the major pharmacologically active compound in the T. camphoratus fruiting body. This invention explored the molecular mechanisms of ZAA in hepatitis treatment.

### Methods

HotLig modelling approach was used to generate the binding model for ZAA with JAK2 and JAK3. JAK2- and JAK3-specific antibody competition analysis was used to demonstrate the interaction between ZAA and JAK2/3. The Con-A-induced C57/BL6 liver injury murine model was used to evaluate the ZAA therapeutic efficacy.

### Results

The computer models showed that ZAA could bind to the hydrophobic pocket of JAK2 and JAK3 exclusively via the H-bond. Further experiments demonstrated that the binding of ZAA to human JH1-JH2 domain could reduce the hybridisation of antibodies to the native JH1-JH2 domain. ZAA could suppress phosphorylation of JAK2 and JAK3 and signaling of downstream molecules. Moreover, ZAA could inhibit the IFN-γ/STAT1/IRF-1 pathway in vivo and in vitro. Furthermore, data show that pre-treatment with ZAA could significantly ameliorate Con A-induced hepatitis in mice.

### Conclusions

The above results strongly suggest that ZAA treatment could block JAK2 and JAK3 activation, and may be a valuable therapeutic approach for the treatment of immune cell induced inflammation.

In the present invention, results of modelling analysis of HotLig [18] clearly show that ZAA could interact with the ATP-binding pocket of JAK2 and JAK3 enzymes through H-bonds, and the association was confirmed by a competition assay using both JAK2- and JAK3-specific antibodies. Binding of ZAA to the JH1-JH2 domain reduced the antibody recognition of the native JH1-JH2 domain. Moreover, ZAA inhibited the STAT1/IRF-1 signaling pathway and the secretion of IFN-γ in vitro and in vivo. Intraperitoneal (i.p.) administration of ZAA protected mice against Con A-induced acute hepatitis. To our best knowledge, this is the first evidence showing that ZAA, a small molecule lanosterol, could inhibit JAK2 and JAK3 phosphorylation. This invention suggests that ZAA can potentially act as a therapeutic agent to protect against immune diseases caused by inflammatory cytokines.

### Materials and Methods

### Cells and Mice

The Jurkat (human T lymphocyte), THP-1 (human monocyte), and HepG2 cell lines were obtained from the Bioresource Collection and Research Center (Hsinchu, Taiwan). Mouse CD4⁺ splenocytes were collected using the CD4⁺-T cell isolation kit (PerkinElmer, UK). Male BALB/c and C57/BL6 mice (8-10-week-old) were obtained from the National Laboratory Animal Center (Taipei, Taiwan). The experimental protocol adhered to the rules of the Animal Protection Act of Taiwan and was approved by the Laboratory Animal Care and Use Committee of the National Cheng Kung University.

### Extraction and Isolation of Fungal Compounds

ZAA was isolated from T. camphoratus as previously described [19, 20]. The compound was dissolved in 40% cyclodextrin (Sigma-Aldrich, St. Louis, Mo.) at a concentration of 2 mg/mL for use as stock solutions, stored at −20° C. and diluted with cell culture medium prior to each experiment. The final concentration of cyclodextrin used in all experiments was below 0.2%.

### Immunoblot Analysis

Jurkat and THP-1 cells (2×10⁶) were treated with various concentrations of ZAA for 3 h, stimulated with predetermined concentrations of IL-2, IL-6, IFN-γ and GM-CSF for 30 min and lysed with RIPA lysis buffer (50 mM Tris-HCl, 150 mM NaCl, 2 mM EDTA, pH 8.0, 1 mM Na₃VO₄, 20 μg/mL leupeptin, 20 μg/mL aprotinin, 1 mM PMSF, 50 mM NaF). Cell lysates were analysed by western blot with primary antibodies against JAK2, pJAK2, JAK3, pJAK3, pSTAT1, pSTAT3, pSTAT5 and β-actin, followed by the appropriate HRP-linked secondary antibodies. Immunoreactive protein bands were detected using an enhanced chemiluminescence (ECL) kit (Pierce Biotechnology, Rockford, Ill.).

### Molecular Docking

Flexible molecular docking was performed using Dock 5.1.1 software [21]. Kollam partial charges were applied to protein models for force field calculation. Energy-optimised three-dimensional coordinates of small molecules were generated using Marvin 5.2.2 (available at http://www.chemaxon.com) and Balloon 0.6 software [22]. In addition, the Gasteiger partial charges for ligands were calculated by OpenBabel 2.2.3 software [23]. The parameters for the Dock program were set to iteratively generate 1000 orientations and 100 conformers in the binding pocket with “anchor size’ of 1. The docked conformers were re-scored and ranked by HotLig to predict the protein-ligand interactions [18]. The figures for molecular modelling were rendered using Chimera and Ligplot software [24, 25]. The electrostatic potentials of protein were calculated using Delphi [26], with default parameter settings in Chimera [24].

### Con A-Induced Hepatitis and Drug Administration

Mice received i.p. administration of ZAA (20 mg/kg) or dexamethasone (1 mg/kg), 1 h prior to i.v. administration of Con A (15 mg/kg). The positive control animals with Con A-induced hepatitis were given the same amount of solvent (0.2% cyclodextrin) i.p., without any drugs. Blood samples were collected from each group for plasma transaminase activities and cytokine expression level determination. Part of the liver and spleen biopsy samples were fixed in 10% formalin, embedded in paraffin for further hematoxylin-eosin staining and the rest of the tissue samples were frozen at −80° C. for further studies.

### Statistical Analysis

Results are presented as means±standard deviation (SD). Statistical differences were analysed using the Student's unpaired t-test or SigmaPlot™ software (Systat). P-values of less than 0.05 were considered statistically significant.

### Results

### Effects of ZAA on Splenic T-Cell Proliferation Induced by Multiple Stimulators In Vitro

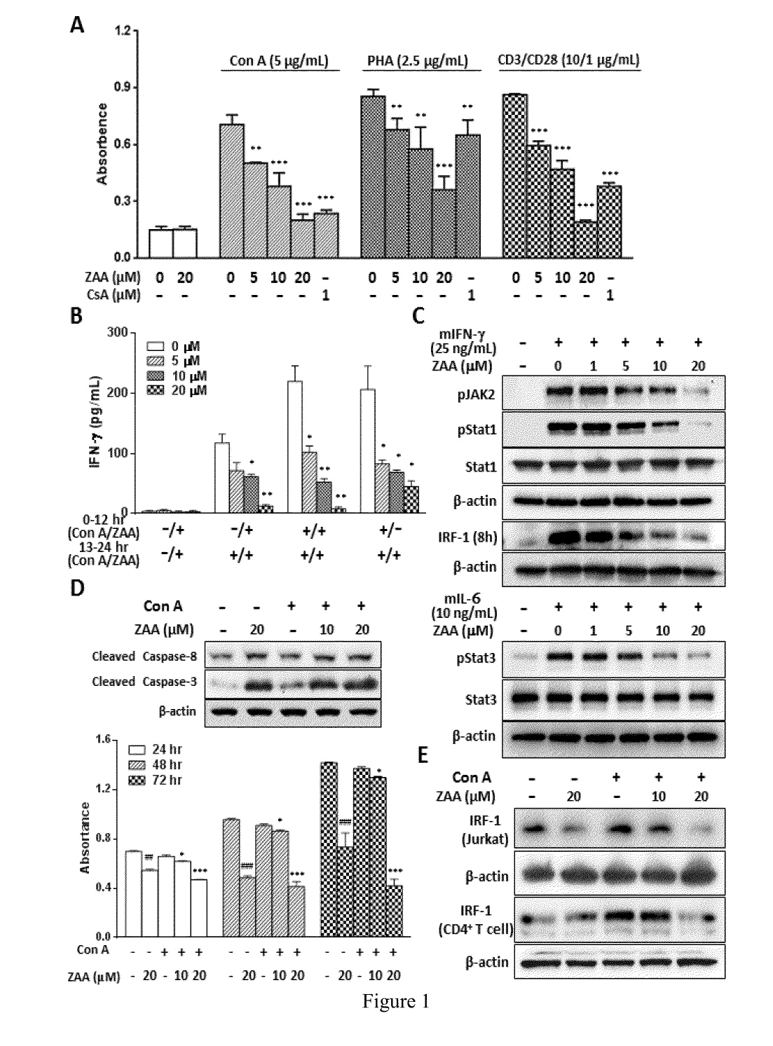

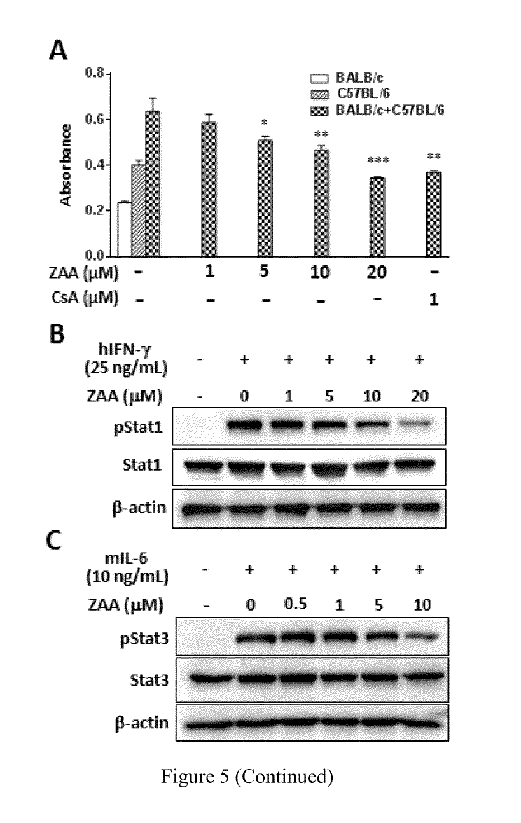

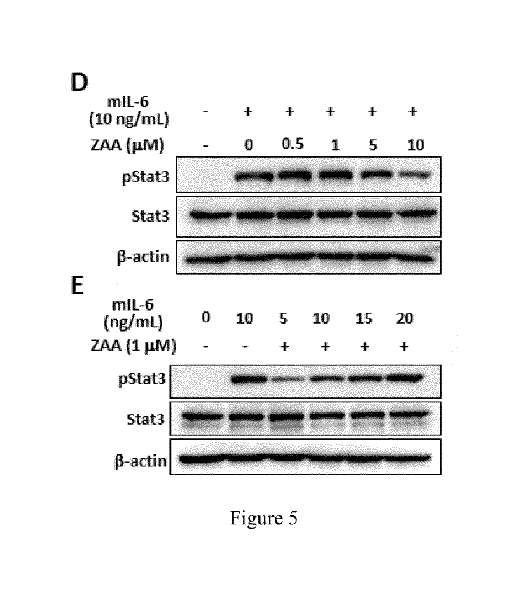

In order to evaluate the immunosuppressive activity of ZAA, Con-A, PHA or anti-CD3/CD28-induced splenocytes were cultured with predetermined ZAA. The results clearly show that ZAA significantly inhibited stimulator-activated T cell proliferation (FIG. 1A) and reduced the Con-A-induced IFN-γ secretion (FIG. 1B), in a concentration-dependent manner. Similarly, the inhibitory effect of ZAA was also shown in a single mixed lymphocytes reaction (FIG. 5A). However, ZAA alone at the tested concentrations (up to 20 μM) did not affect the proliferation of splenocytes, nor was IFN-γ production ability observed. These results suggest that ZAA inhibited lymphocyte activation induced by various stimulators.

ZAA Suppressed IFN-γ/pSTAT1 and IL-6/pSTAT3 Signaling Pathway in Splenocytes

To further analyse the mechanism of ZAA-mediated inhibition of IFN-γ signaling in splenocytes, the phosphorylation status of STAT1 and IRF-1 was measured by immunoblot analysis. Incubation of splenocytes and Jurkat cells with IFN-γ for 30 min resulted in a marked enhancement of STAT1 tyrosine phosphorylation (FIG. 1C and FIG. 5B), while ZAA-co-treatment could completely inhibit the Tyr701 phosphorylation of STAT1. A similar inhibitory effect of ZAA was observed on IRF-1, a downstream molecule of STAT, in a concentration-dependent manner. Moreover, ZAA also could suppress the IL-6-induced Tyr705 phosphorylation of STAT3 in splenocytes (FIG. 1C) and RAW264.7 cells (FIG. 5C). Furthermore, the inhibitory effect of ZAA toward STAT activation could be reversed by high dose IFN-γ and IL-6 treatment (FIGS. 5D and 5E).

To determinate whether ZAA inhibited specifically activated CD4⁺-T cells, we used immortalised human CD4⁺-T lymphocytes (Jurkat cells) to evaluate the cell viability by MTS assay. As shown in FIG. 1D, ZAA remarkably inhibited Jurkat cell proliferation without or with Con A treatment. The increase in cleaved pro-caspase-3 and pro-caspase-8 was also observed at the end of the 24 h ZAA-treatment. This result also indicate that the inhibitory effect of Con A-induced cell proliferation was more obvious than non-Con A-induced cell proliferation with ZAA treatment, over time. To distinguish whether ZAA possesses differential cytotoxicity toward the activated and normal CD4⁺ lymphocyte, Jurkat cells and CD4⁺ splenocytes were exposed to ZAA without or with Con A treatment, and the IRF-1 molecule was evaluated by immunoblot analysis. As shown in FIG. 1E, ZAA (20 μM) suppressed IRF-1 expression in Jurkat but not CD4⁺ splenocytes, compared to the vehicle-treated cells. These results suggest that ZAA could suppress the activating CD4⁺ cells, such as Jurkat cells, but not normal cells. In other words, ZAA was safe in normal cells, but not in rapidly proliferating cells.

### Prediction of ZAA-Binding Targets

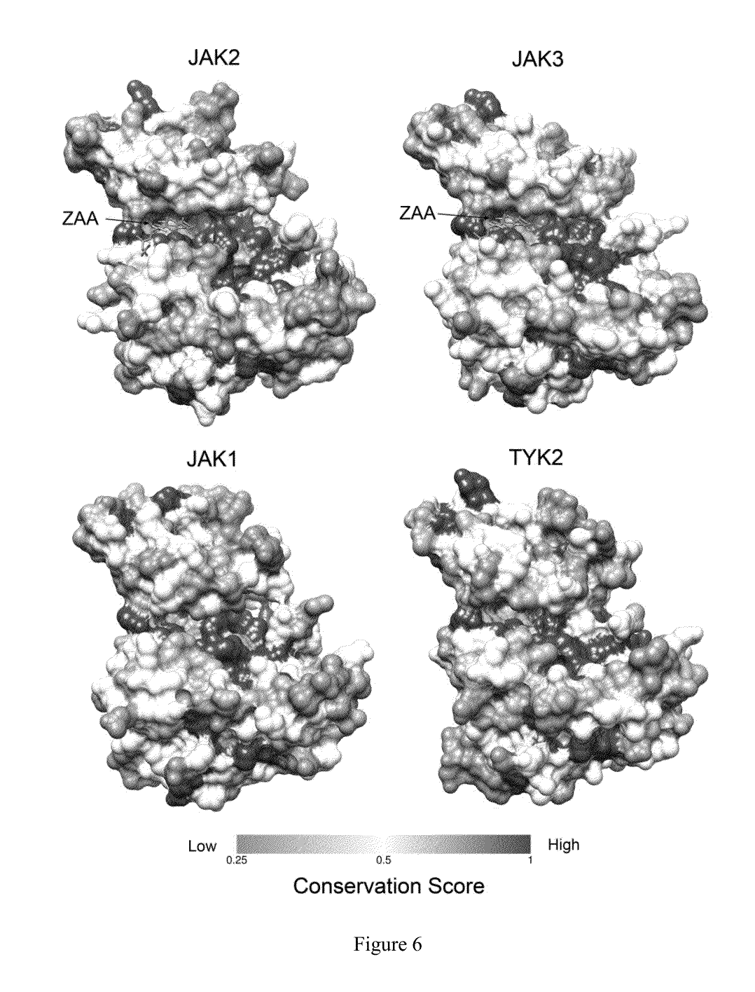

In order to predict the binding targets of ZAA, the structures of IFN-γ, STAT1, IRF-1 and JAK family (JAK1, JAK2, JAK3 and TYK2) were surveyed. Only the protein structures of the JAK family were found to possess significant pockets for the binding of ZAA. Subsequently, the potential interactions between ZAA and the proteins of the JAK family were investigated through a molecular docking study. As described in ‘Methods’, the docking conformers of ZAA were sampled against the protein structures using the Dock software [21] and then these conformers were scored and ranked by HotLig [18] to predict the interactions of ZAA with each JAK kinase. As indicated in FIG. 6, the ZAA was found to bind the substrate-binding sites on JAK2 and JAK3 rather than JAK1 or TYK2. The conservation of amino acid sequences among these four kinases was further analysed. The amino acid residues in the regions of substrate-binding sites were highly conserved in these four kinases. However, the binding pockets on JAK1 and TYK2 were probably too narrow for ZAA to access.

ZAA Interacts with the Hydrophobic Pocket of JAK2 and JAK3 to Block Tyrosine Kinase Phosphorylation

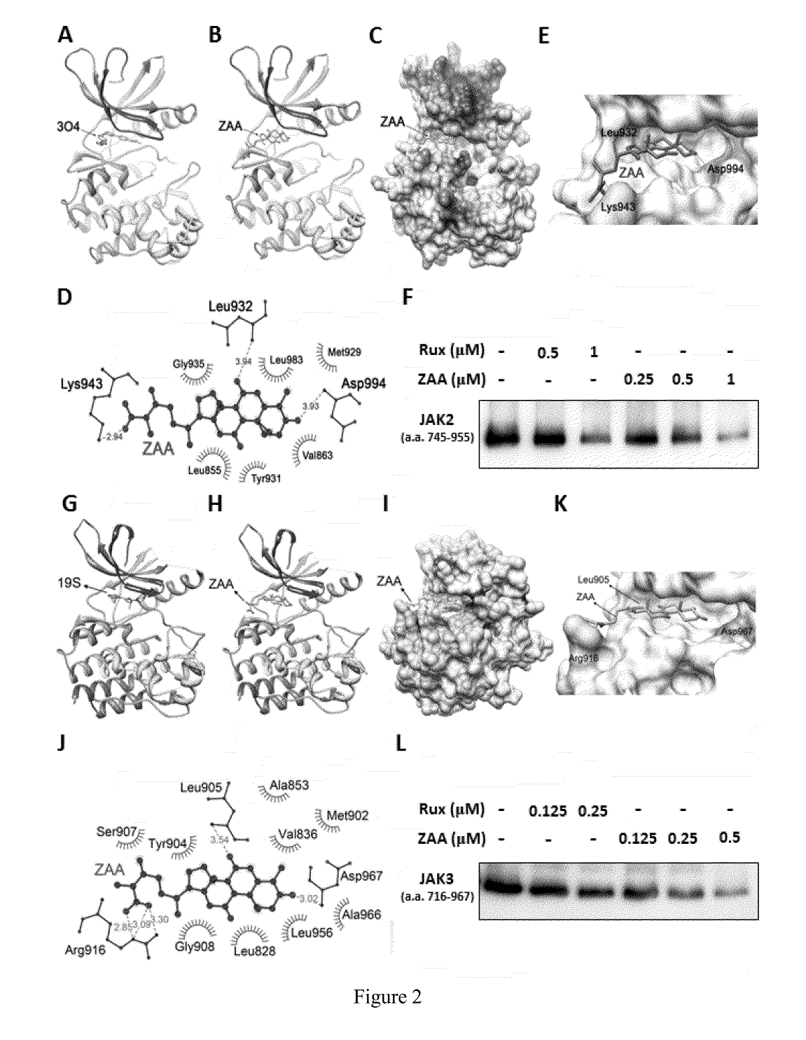

As shown in FIG. 2A, a structural model of human JAK2 kinase (PDB code: 4BBE) [27], which is complexed with a selective inhibitor, 304 [28], was used for the molecular docking study. As described in ‘Methods’, the docking conformers of ZAA were generated against the 304-binding pocket by the Dock software [21] and rescored by the HotLig to predict the interactions of ZAA with JAK2 kinase. The calculated binding energy score of ZAA was −28.52, which was as good as that of 304 (−28.46) (Table 1). The predicted binding pose of ZAA is shown in FIG. 2B. The electrostatic potentials on the protein surface of JAK2 kinase were also calculated using the Delphi program [26]. As shown in FIG. 2C, the negatively charged potential is coloured in red whereas the positively charged potential is coloured in blue. The binding pocket of ZAA mainly presents a white colour, indicating that the pocket is composed of hydrophobic amino acid residues. The detailed interactions between the ZAA and the binding pocket are shown in FIG. 2D. The ZAA possesses one significant H-bond with the amino acid Lys943 of JAK2 kinase (H-bond length of 2.94 Å). In addition, two potential weak H-bonds (H-bond lengths were 3.93-3.94 Å) were found to interact with the amino acids Leu932 and Asp994. The amino acid residues, Gly935, Leu983, Va1863, Tyr931 and Leu855, of JAK2 kinase were found to interact with the ZAA through hydrophobic contacts (FIG. 2D). FIG. 2E shows a closer view of the ZAA-binding pocket in the 3-D model. The positions of three amino acids, Lys943 (light blue), Leu932 (yellow) and Asp 994 (orange), which form H-bonds with the ZAA, are labelled to indicate their interactions with the ZAA molecule. We next performed immunoelectrophoresis with the anti-JAK2 antibody, monoclonal antibody against JAK2 amino acids 745-955. Binding of ZAA to the human JH1-JH2 domain reduced the antibody recognition to the native JH1-JH2 domain, similar to the effect of ruxolitinib binding (FIG. 2F).

TABLE 1
HotLig scores of complex structures of the JAK family with
ZAA or their native ligands.

| | PDB codes of protein structures | | | |
|---|---|---|---|---|
| | | | | 3LXP |
| Ligands | 4KF6 (JAK1) | 4BBE (JAK2) | 4HVI (JAK3) | (TYK2) |
| ZAA | −21.67 | −28.52 | −27.38 | −23.25 |
| 4KF6-OUJ | −26.82 | — | — | — |
| 4BBE-3O4 | — | −28.46 | — | — |
| 4HVI-19S | — | — | −28.33 | — |
| 3LXP-IZA | — | — | — | −27.47 |

Similarly, FIG. 2G shows that the human JAK3 kinase (PDB code: 4HVI) in complex with a JAK3-selective inhibitor, 19S, whose binding energy score for ZAA was −27.38, which was as good as that of the 19S (−28.33) (Table 1). The predicted binding pose of ZAA is shown in FIG. 2H. As shown in FIG. 2I, the negatively charged potential is coloured red whereas the positively charged potential is coloured blue. The ZAA possesses five significant H-bonds with the amino acid Arg916 of JAK3 kinase (H-bond length of 2.85, 3.09 and 3.30 Å), Leu905 of JAK3 kinase (H-bond length of 3.54 Å) and Asp967 of JAK3 kinase (H-bond length of 3.02 Å) (FIG. 2J). FIG. 2K presents a closer view of the ZAA-binding pocket on the 3-D model. We also performed immunoelectrophoresis with anti-JAK3 antibody, polyclonal antibody against JAK2 amino acids 716-967. Binding of ZAA to the human JH1-JH2 domain reduced the antibody recognition of the native JH1-JH2 domain, similar to the effect of ruxolitinib binding (FIG. 2L).

Thus, the molecular docking results suggest that the ZAA might predominantly target JAK2 and JAK3 kinases. A similar approach was used for IFN-γ, STAT1 and IRF-1; however, no significant binding pocket for ZAA was identified.

### ZAA Inhibited the Phosphorylation of JAK2 and JAK3 and Downstream Signaling

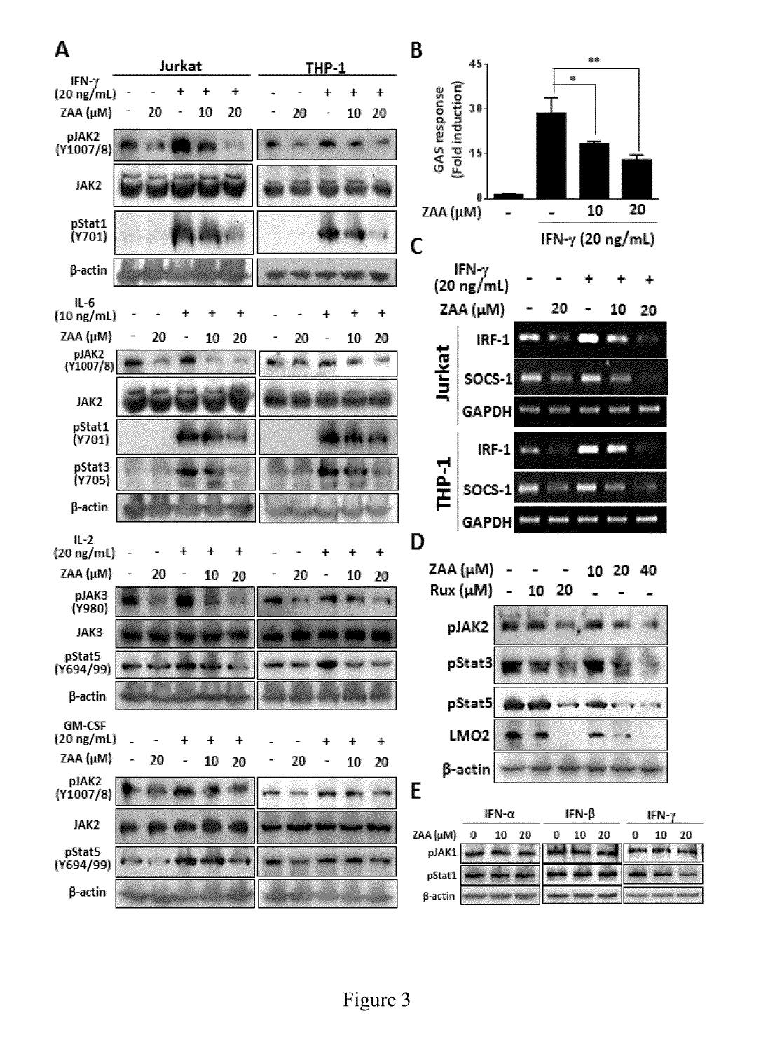

To determine whether ZAA inhibits JAK2 and JAK3 signaling through binding to the hydrophobic pocket of the JAKs, we investigated JAKs and STATs phosphorylation in Jurkat and THP-1 cells that had been treated with the tyrosine phosphatase inhibitors for evaluating the effect [29, 30]. As demonstrated by immunoblot analysis (FIG. 3A), ZAA potently inhibited IFN-γ-induced JAK2/STAT1, IL-6 induced JAK2/STAT1 and JAK2/STAT3, IL-2 induced JAK3/STAT5 and GM-CSF induced JAK2/STAT5 auto-phosphorylation and phosphorylation. Furthermore, to investigate the inhibitory role of ZAA in IFN-γ-stimulated interferon-gamma activated sequence (GAS) signaling, we detected its effect on the transactivation of GAS. FIG. 3B showed that ZAA inhibited GAS-mediated transactivation in Jurkat cells, as revealed by the luciferase reporter assay. In addition, IFN-γ treatment stimulated IRF-1 and suppressor of cytokine signaling 1 (SOCS-1) transcription, which was significantly prevented by ZAA (FIG. 3C). As JAK2/STAT3 signaling has a role in human hepatoma HepG2 cell proliferation [31], JAK2 activation promotes recruitment to the receptor complex of STAT3 and STAT5 [32], and LMO2 expression [10]. Therefore, ZAA activity toward the SATA3/5 signaling pathway was compared with ruxolitinib, the JAK1/JAK2 inhibitor [33]. As shown in FIG. 3D, both ZAA and ruxolitinib could effectively inhibit the phosphorylation of JAK2, STAT3 and STAT5, and LMO2 expression at the dose of 20 μM after 48 h culture in HepG2 cells. These results suggest that ZAA could suppress JAK2 and JAK3-induced downstream signaling by blocking JAK2 and JAK3 phosphorylation. By contrast, we also determined whether the IFN-α and IFN-β-induced STAT1 phosphorylation via JAK1 and TYK2 was suppressed by ZAA. As shown in FIG. 3E, ZAA could not inhibit IFN-α and IFN-β to induce STAT1 phosphorylation, but did inhibit IFN-γ. JAK1 phosphorylation was not suppressed by ZAA at 20 μM.

### ZAA Pre-Treatment Effectively Attenuates Con A-Induced Liver Injury

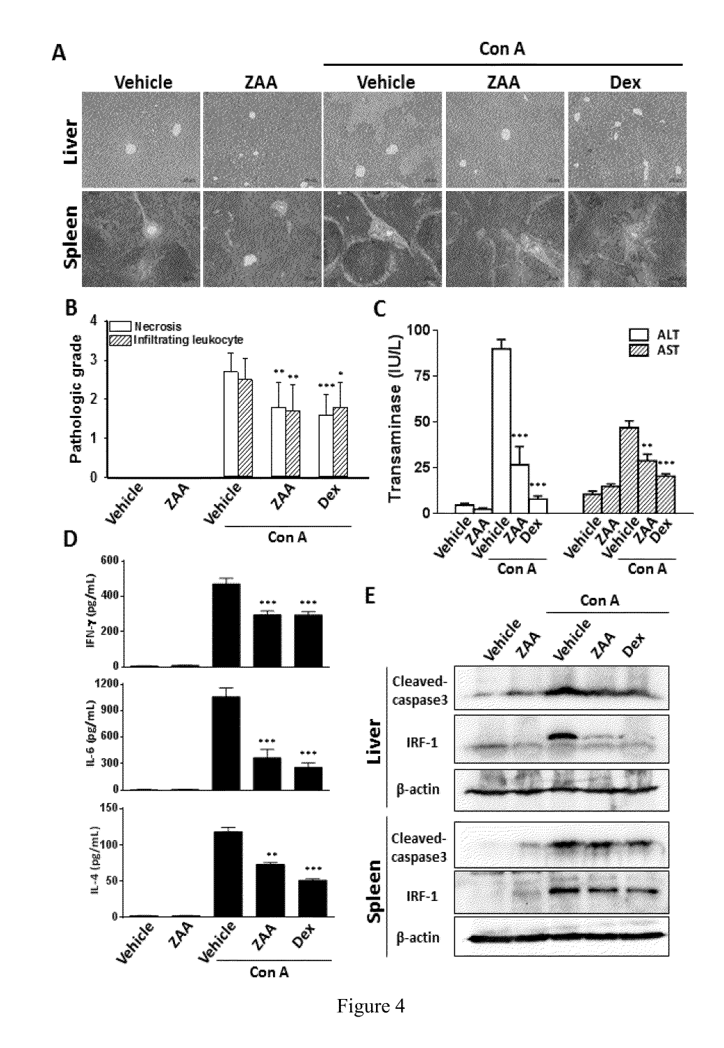

A previous study demonstrated that up-regulation of expression of the downstream target of IFN-γ, IRF-1, plays a critical role in Con A-mediated liver injury [34], and disruption of the IRF-1 gene expression or activation could protect mortality associated with injection of Con A [35]. To determine whether ZAA could promote the hepato-protective effect toward Con A-induced T-cell-mediated acute hepatitis, mice were pretreated with ZAA. As shown in FIG. 4A, massive cell necrosis with cytoplasmic swelling and infiltration of leukocytes in the liver biopsy of Con A-treated mice was observed after an 8-h treatment; splenomegaly was used as the activated lymph organ. However, pre-treatment with 20 mg/kg of ZAA could markedly reduce the extent of liver damage, with minimal leukocyte infiltration. The pathologic grades also showed the significant preventative effects of ZAA on necrosis and infiltration of leukocytes (FIG. 4B), and alleviated Con A-induced hepatitis phenotype was almost completely recovered based on the evaluation of ALT and AST serum levels (FIG. 4C). The influence of ZAA on cytokine production in Con A-induced hepatitis was also determined. As shown in FIG. 4D, ZAA pre-treatment reduced serum levels of IFN-γ by 37%, IL-6 by 66% and IL-4 by 38%, 8 h after Con A injection. Results presented in FIG. 4E indicate that 20 mg/kg of ZAA pre-treatment significantly reduced caspase 3 activity and IRF-1 expression in the liver and spleen tissues. Similar results were observed in the dexamethasone-treated mice and showed significantly attenuated Con A-induced acute hepatitis (FIG. 4). These results suggest that ZAA protected the liver cells against apoptosis induced by the IFN-γ/IRF-1 pathway.

### ZAA Inhibited IFN-γ Activated Cells in the Progression of Con A-Induced Hepatitis

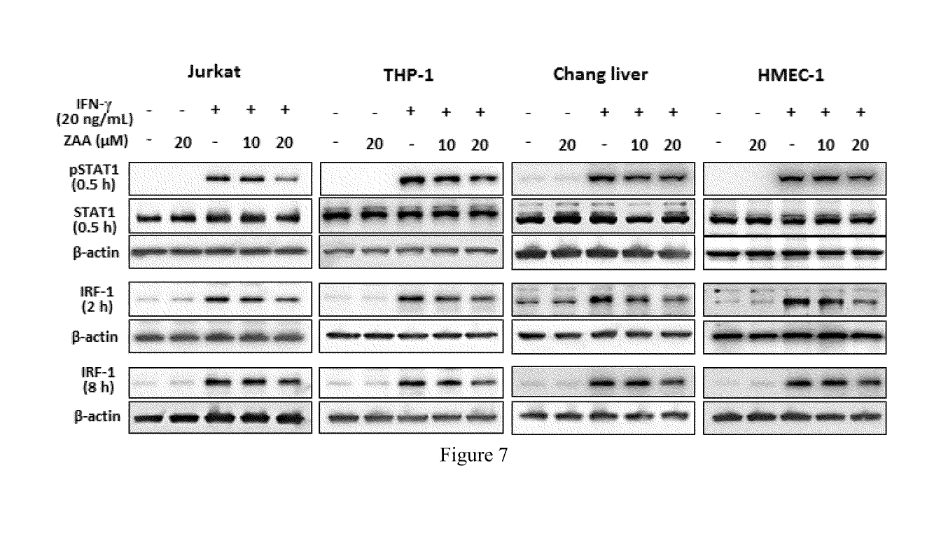

To further understand whether ZAA inhibition of the IFN-γ activated signaling phenomena generally occurred in other hepatic cell types, IFN-γ-pre-treated HMEC-1 and Chang liver cells were used to evaluate the ZAA inhibitory effect. As shown in FIG. 7, IFN-γ activated pSTAT1 and IRF-1 (2 h and 8 h) in Jurkat, THP-1, HMEC-1 and Chang liver cells were suppressed by ZAA pre-treatment. These results suggest that ZAA suppression of hepatitis through the inhibition of IFN-γ/STAT1/IRF-1 signaling pathway might be a general mechanism in hepatic cells.

### Discussion

The mechanisms of action of ZAA are complex, including integrating pro-apoptotic effects, inhibition of proliferation and DNA damage. Although much information is available concerning the anti-cancer effect of ZAA, relatively few studies have been conducted documenting the molecular mechanisms of ZAA inhibition of the growth of activated immune cells, which are not well elucidated yet. In this invention, our results clearly present important observations: (1) ZAA inhibited the activation of JAK2/STATs and JAK3/STAT5 in a dose-dependent manner, and simultaneously down-regulated the downstream genes; (2) ZAA-induced apoptosis was observed in activated and highly proliferative T-cell leukaemia but not normal immune cells; (3) ZAA alleviated Con A-induced hepatitis by suppressing JAK2/STAT1/IRF1 signaling pathway, both in vitro and in vivo. The above findings suggest that ZAA might be potentially useful in treating inflammatory disorders.

The JAK1/JAK2 inhibitor ruxolitinib has been approved by the FDA for treatment of constitutively activated JAK2 myelofibrosis [36]. A previous study showed that ruxolitinib interacts with the Met-929, Tyr-931 and Gly-935 of JH1-JH2 domain of JAK2 [37], suggesting that this amino acid region is critical for JAK2 kinase activity. According to our computer modelling analysis results, these amino acids are close to Leu-932, Lys-943 and Asp-994 that can form H-bonds with the ZAA. Moreover, further analysis showed that the three H-bonds in ZAA interact with Arg-916, one H-bond with Leu905 and one H-bond with Asp967 of JAK3 kinase. By contrast, the interaction between ZAA and JAK2 or JAK3 is much better than JAK1 and TYK2, suggesting that it could be used for the treatment of myeloproliferative disorders, and inhibitors as immunosuppressants for organ transplants and autoimmune diseases.

Our previous study has demonstrated that ZAA is a LPS antagonist of NF-κB activation in response to inflammatory stimuli. Whether suppression of STAT3 by ZAA is associated with its observed inhibitory effects on NF-κB pathway needs further investigation. Yu et al., have demonstrated that the p65 subunit of NF-κB closely communicates with STAT3 [38], but in general the activation of STAT3 and NF-κB are dependent on different cytokines. While IL-6 is a major activator of STAT3, LPS is also a potent activator of NF-κB. The activation of JAK2 kinase has also been shown to be necessary for erythropoietin-induced NF-κB activation [39]. Thus, it is possible that suppression of JAK2 activation is the potential link for inhibition of both NF-κB and STAT3 activation by ZAA.

STAT protein activation in the liver is critical for anti-viral defence against hepatitis viral infection and for controlling injury, inflammation and tumorigenesis [40]. IFN-γ activation of STAT1 directly induced hepatocyte apoptosis, resulting in apoptosis-associated liver inflammation [41, 42]. Transgenic mice over-expressing STAT1 in T cells are more susceptible to Con A-induced hepatitis, suggesting that inhibition of STAT1 could suppress liver inflammation [41]. Our results clearly demonstrate that ZAA could inhibit Con A-activated IFN-γ secretion by suppressing cell proliferation, and IFN-γ stimulated IRF-1 expression by blocking JAK2 phosphorylation, both in vitro and in vivo. Thus ZAA could be a useful therapeutic option against IFN-γ/STAT1-activated inflammatory disorders.

### Supplemental Materials

### Reagents

Concanavaline A, cyclosporine A, dexamethasone, mitomycin C and cyclodextrin were purchased from Sigma-Aldrich (St. Louis, Mo.). Ruxolitinib was from Biochempartner (ShangHai, China). Trizol reagent and phytohemagglutinin (PHA) were from Invitrogen. ELISA kits for mouse IFN-γ, IL-6 and IL-4 were purchased from R&D Systems (Minneapolis, Minn.). IRF-1, caspase-8, pJAK1, JAK2, pJAK2, JAK3 (C-21), pJAK3, STAT1, STAT3, pSTAT5 and LMO2 antibodies were purchased from Santa Cruz Biotechnology Inc. (Santa Cruz, Calif.). Antibody against JAK2 (aa, 745-955) was from Abcam (Cambridge, Mass.) and the antibody against JAK3 (aa, 716-967) was from Cloud-Clone (Houston, US). Human JAK2 JH1-JH2 and JAK3 JH1-JH2 was from Life Technologies (California, US). Recombinant human IFN-α, IFN-β, IFN-γ, IL-6, IL-2 and GM-CSF were provided by Peprotech (Rocky Hill, N.J.). Antibodies against caspase-3, pSTAT1 and pSTAT3 were from Cell Signaling. Antibodies against CD3 and CD28 were from BD PharMingen (San Diego, Calif.).

### Mixed Lymphocyte Reaction Assay

BALB/c splenocytes (4×10⁵) were treated with mitomycin C dissolved in cyclodextrin (25 μg/mL) for 1 h and then co-cultured with C57/BL6 splenocytes (4×10⁵) in the absence or presence of various concentrations of ZAA for 72 h. The cell number of total lymphocytes was measured using the CellTiter kit.

### Cell Viability Assay

Primary mouse splenocytes were cultured in 96-well plates at a density of 4×10⁵ cells/well in 10% FBS RPMI 1640 medium and activated with three different stimulators (Con A: 5 μg/mL; PHA: 2.5 μg/mL; Anti-CD3/CD28: 10/1 μg/mL) at 37° C. in 5% CO₂/air. After 48 h incubation, the indicated concentration of ZAA was included for a further 48-h culture. Jurkat cells were plated in 96-well plates at a density of 2×10³ cells/well in 10% FBS RPMI 1640 medium and co-cultured with the indicated concentrations of ZAA and Con A (5 μg/mL) for 24 h, 48 h and 72 h. The cell proliferation was evaluated by MTS assay with a CellTiter kit (Promega, Madison, Wis.).

### IFN-γ Secretion Analysis

Primary mouse splenocytes were plated at 1×10⁵ cells/well with 10% FBS RPMI medium in 96-well plates. Four different experimental conditions were tested. First, cells were treated with vehicle or various concentrations of ZAA for 24 h. Second, cells were treated with vehicle or various concentration of ZAA for 12 h, followed by Con A activation for 12 h. Third, cells were treated with vehicle or various concentrations of ZAA and Con A for 24 h. Fourth, cells were activated with Con A for 12 h and treated with vehicle or various concentrations of ZAA. All experiments were conducted at 37° C. After a total 24 h of incubation, supernatant IFN-γ concentrations were measured by ELISA kit following the manufactures instructions.

### Native PAGE

For in vitro binding analyses, pre-determined amounts of ruxolitinib or ZAA were sonicated for 3 min and incubated with human JAK2 JH1-JH2 and JAK3 JH1-JH2 at 37° C. for 3 h. Samples were loaded onto native PAGE gels for electrophoresis, and the levels of free JH1-JH2 domain were measured by immunoblotting. Signals were detected via ECL following the manufacture's suggestion.

### Reverse Transcription-Polymerase Chain Reaction

Total RNA was extracted using the Trizol reagent according to the manufacturer's instructions. IRF-1, SOCS-1 and glyceraldehyde 3-phosphate GAPDH mRNA expression levels were detected by RT-PCR.

### Reporter Assay for GAS Transactivation Activity

The serum-containing medium of logarithmic growth Jurkat cells was replaced with serum-free RPMI, and the cells were transfected with p-GAS-Luc (0.5 μg/mL) plasmids using the Neon Transfection System (Invitrogen). Four hours later, the serum-free medium was replaced with RPMI containing 10% FBS. Twenty-four hours post-transfection, cells were co-cultured in serum-free RPMI without or with 10 or 20 μM of ZAA for 3 h, followed by IFN-γ (final concentration: 20 ng/mL) for 24 h. Luciferase reporter activity was measured according to the manufacturer's recommendation.

### Analysis of Plasma Transaminase Activities

Liver injury was quantified by determination of alanine aminotransferase (ALT) and aspartate aminotransferase (AST) activities in serum, according to the Reitman-Frankel method.

### Immunoblot Analysis

Jurkat, Raw264.7, THP-1 cells, Chang liver and HMEC-1 (human microvascular endothelial cell) (2×10⁶) were treated with various concentrations of ZAA for 3 h, stimulated with predetermined concentrations of IL-6 and IFN-γ for 30 min and lysed with RIPA lysis buffer (50 mM Tris-HCl, 150 mM NaCl, 2 mM EDTA, pH 8.0, 1 mM Na₃VO₄, 20 μg/mL leupeptin, 20 μg/mL aprotinin, 1 mM PMSF, 50 mM NaF). Cell lysates were analysed by immunoblotting with primary antibodies against pSTAT1, pSTAT3, IRF-1 and β-actin, followed by the appropriate HRP-linked secondary antibodies. Immunoreactive protein bands were detected using an enhanced chemiluminescence (ECL) kit (Pierce Biotechnology, Rockford, Ill.).

### REFERENCES

[1] Ghoreschi K, Laurence A, O'Shea J J. Janus kinases in immune cell signaling. Immunological reviews 2009; 228:273-287.
[2] Kiu H, Nicholson S E. Biology and significance of the JAK/STAT signaling pathways. Growth Factors 2012; 30:88-106.
[3] Darnell J E, Jr., Kerr I M, Stark G R. Jak-STAT pathways and transcriptional activation in response to IFNs and other extracellular signaling proteins. Science 1994; 264:1415-1421.
[4] Kisseleva T, Bhattacharya S, Braunstein J, Schindler C W. Signaling through the JAK/STAT pathway, recent advances and future challenges. Gene 2002; 285:1-24.
[5] Narazaki M, Witthuhn B A, Yoshida K, Silvennoinen O, Yasukawa K, Ihle J N, et al. Activation of Jak2 Kinase Mediated by the Interleukin-6 Signal Transducer Gp130. Proceedings of the National Academy of Sciences of the United States of America 1994; 91:2285-2289.
[6] Silvennoinen O, Ihle J N, Schlessinger J, Levy D E. Interferon-Induced Nuclear Signaling by Jak Protein-Tyrosine Kinases. Nature 1993; 366:583-585.
[7] Padron E, Painter J S, Kunigal S, Mailloux A W, McGraw K, McDaniel J M, et al. G M-CSF-dependent pSTAT5 sensitivity is a feature with therapeutic potential in chronic myelomonocytic leukemia. Blood 2013; 121:5068-5077.
[8] Zhao Y M, Wagner F, Frank S J, Kraft A S. The Amino-Terminal Portion of the Jak2 Protein-Kinase Is Necessary for Binding and Phosphorylation of the Granulocyte-Macrophage Colony-Stimulating Factor-Receptor Beta(C) Chain. Journal of Biological Chemistry 1995; 270:13814-13818.
[9] Delhommeau F, Pisani D F, James C, Casadevall N, Constantinescu S, Vainchenker W. Oncogenic mechanisms in myeloproliferative disorders. Cellular and molecular life sciences: CMLS 2006; 63:2939-2953.
[10] Dawson M A, Bannister A J, Gottgens B, Foster S D, Bartke T, Green A R, et al. JAK2 phosphorylates histone H3Y41 and excludes HPlalpha from chromatin. Nature 2009; 461:819-822.
[11] Shi M, Lin T H, Appell K C, Berg L J. Janus-kinase-3-dependent signals induce chromatin remodeling at the Ifng locus during T helper 1 cell differentiation. Immunity 2008; 28:763-773.
[12] Thomis D C, Berg L J. The role of Jak3 in lymphoid development, activation, and signaling. Current Opinion in Immunology 1997; 9:541-547.
[13] Johnston J A, Kawamura M, Kirken R A, Chen Y Q, Blake T B, Shibuya K, et al. Phosphorylation and Activation of the Jak-3 Janus Kinase in Response to Interleukin-2. Nature 1994; 370:151-153.
[14] Yoneda K, Osaki T, Yamamoto T. IL-2 signaling in T and natural killer (NK) cells associated with their class I-non-restricted killing activity. Clinical and Experimental Immunology 1996; 106:179-186.
[15] Chen C H, Yang S W, Shen Y C. New steroid acids from Antrodia cinnamomea, a fungal parasite of Cinnamomum micranthum. J Nat Prod 1995; 58:1655-1661.
[16] Liu D Z, Liang H J, Chen C H, Su C H, Lee T H, Huang C T, et al. Comparative anti-inflammatory characterization of wild fruiting body, liquid-state fermentation, and solid-state culture of Taiwanofungus camphoratus in microglia and the mechanism of its action. J Ethnopharmacol 2007; 113:45-53.
[17] Rao Y K, Fang S H, Tzeng Y M. Evaluation of the anti-inflammatory and anti-proliferation tumoral cells activities of Antrodia camphorata, Cordyceps sinensis, and Cinnamomum osmophloeum bark extracts. J Ethnopharmacol 2007; 114:78-85.
[18] Wang S H, Wu Y T, Kuo S C, Yu J. HotLig: A Molecular Surface-Directed Approach to Scoring Protein-Ligand Interactions. Journal of chemical information and modeling 2013; 53:2181-2195.
[19] Wu S J, Leu Y L, Chen C H, Chao C H, Shen D Y, Chan H H, et al. Camphoratins A-J, potent cytotoxic and anti-inflammatory triterpenoids from the fruiting body of Taiwanofungus camphoratus. J Nat Prod 2010; 73:1756-1762.
[20] Shi L S, Chao C H, Shen D Y, Chan H H, Chen C H, Liao Y R, et al. Biologically active constituents from the fruiting body of Taiwanofungus camphoratus. Bioorg Med Chem 2011; 19:677-683.
[21] Moustakas D T, Lang P T, Pegg S, Pettersen E, Kuntz I D, Brooijmans N, et al. Development and validation of a modular, extensible docking program: DOCK 5. Journal of computer-aided molecular design 2006; 20:601-619.
[22] Vainio M J, Johnson M S. Generating conformer ensembles using a multiobjective genetic algorithm. Journal of chemical information and modeling 2007; 47:2462-2474.
[23] Guha R, Howard M T, Hutchison G R, Murray-Rust P, Rzepa H, Steinbeck C, et al. The Blue Obelisk-interoperability in chemical informatics. Journal of chemical information and modeling 2006; 46:991-998.
[24] Pettersen E F, Goddard T D, Huang C C, Couch G S, Greenblatt D M, Meng E C, et al. UCSF Chimera—a visualization system for exploratory research and analysis. Journal of computational chemistry 2004; 25:1605-1612.
[25] Wallace A C, Laskowski R A, Thornton J M. LIGPLOT: a program to generate schematic diagrams of protein-ligand interactions. Protein engineering 1995; 8:127-134.
[26] Meng E C, Pettersen E F, Couch G S, Huang C C, Ferrin T E. Tools for integrated sequence-structure analysis with UCSF Chimera. BMC bioinformatics 2006; 7:339.
[27] Rocchia W, Alexov E, Honig B. Extending the applicability of the nonlinear Poisson-Boltzmann equation: Multiple dielectric constants and multivalent ions. J Phys Chem B 2001; 105:6507-6514.
[28] Forsyth T, Kearney P C, Kim B G, Johnson H W B, Aay N, Arcalas A, et al. SAR and in vivo evaluation of 4-aryl-2-aminoalkylpyrimidines as potent and selective Janus kinase 2 (JAK2) inhibitors. Bioorg Med Chem Lett 2012; 22:7653-7658.
[29] Rahmouni S, Delacroix L, Liu W H, Tautz L. Evaluating effects of tyrosine phosphatase inhibitors on T cell receptor signaling. Methods Mol Biol 2013; 1053:241-270.
[30] Butturini E, Cavalieri E, de Prati A C, Darra E, Rigo A, Shoji K, et al. Two naturally occurring terpenes, dehydrocostuslactone and costunolide, decrease intracellular GSH content and inhibit STAT3 activation. PLoS One 2011; 6:e20174.
[31] Liu Y Y, Zheng Q, Fang B, Wang W, Ma F Y, Roshan S, et al. Germacrone induces apoptosis in human hepatoma HepG2 cells through inhibition of the JAK2/STAT3 signaling pathway. Journal of Huazhong University of Science and Technology Medical sciences=Hua zhong ke ji da xue xue bao Yi xue Ying De wen ban=Huazhong keji daxue xuebao Yixue Yingdewen ban 2013; 33:339-345.
[32] Levine R L, Pardanani A, Tefferi A, Gilliland D G Role of JAK2 in the pathogenesis and therapy of myeloproliferative disorders. Nature reviews Cancer 2007; 7:673-683.
[33] Verstovsek S. Ruxolitinib: an oral Janus kinase 1 and Janus kinase 2 inhibitor in the management of myelofibrosis. Postgraduate medicine 2013; 125:128-135.
[34] Jaruga B, Hong F, Kim W H, Gao B. IFN-gamma/STAT1 acts as a proinflammatory signal in T cell-mediated hepatitis via induction of multiple chemokines and adhesion molecules: a critical role of IRF-1. American journal of physiology Gastrointestinal and liver physiology 2004; 287:G1044-1052.
[35] Senaldi G, Shaklee C L, Guo J, Martin L, Boone T, Mak T W, et al. Protection against the mortality associated with disease models mediated by TNF and IFN-gamma in mice lacking IFN regulatory factor-1. J Immunol 1999; 163:6820-6826.
[36] Ostojic A, Vrhovac R, Verstovsek S. Ruxolitinib: a new JAK1/2 inhibitor that offers promising options for treatment of myelofibrosis. Future oncology 2011; 7:1035-1043.
[37] Zhou T, Georgeon S, Moser R, Moore D J, Caflisch A, Hantschel 0. Specificity and mechanism-of-action of the JAK2 tyrosine kinase inhibitors ruxolitinib and SAR302503 (TG101348). Leukemia 2013.
[38] Yu Z, Zhang W, Kone B C. Signal transducers and activators of transcription 3 (STAT3) inhibits transcription of the inducible nitric oxide synthase gene by interacting with nuclear factor kappaB. The Biochemical journal 2002; 367:97-105.
[39] Digicaylioglu M, Lipton S A. Erythropoietin-mediated neuroprotection involves cross-talk between Jak2 and NF-kappaB signaling cascades. Nature 2001; 412:641-647.
[40] Gao B, Wang H, Lafdil F, Feng D. STAT proteins—key regulators of anti-viral responses, inflammation, and tumorigenesis in the liver. Journal of hepatology 2012; 57:430-441.
[41] Siebler J, Wirtz S, Klein S, Protschka M, Blessing M, Galle P R, et al. A key pathogenic role for the STAT1/T-bet signaling pathway in T-cell-mediated liver inflammation. Hepatology 2003; 38:1573-1580.
[42] Kim W H, Hong F, Radaeva S, Jaruga B, Fan S, Gao B. STAT1 plays an essential role in LPS/D-galactosamine-induced liver apoptosis and injury. American journal of physiology Gastrointestinal and liver physiology 2003; 285:G761-768.

1. A method of treating a subject having a JAK-associated disorder, which comprises administering to said subject in need of said treatment of Zhankuic acid A or a pharmaceutically acceptable salt thereof.

2. The method of claim 1, wherein the JAK-associated disorder is a myeloproliferative disorder.

3. The method of claim 2, wherein the myeloproliferative disorder is polycythemia vera (PV), essential thrombocythemia (ET), myeloid metaplasia with myelofibrosis (MMM), chronic myelogenous leukemia (CML), chronic myelomonocytic leukemia (CMML), hypereosinophilic syndrome (HES), or systemic mast cell disease (SMCD).

4. The method claim 1, wherein the JAK-associate disorder is an immune disorder caused by organ transplant rejection.

5. The method claim 1, wherein the JAK-associate disorder is an autoimmune disease.

6. The method claim 1, wherein the JAK-associate disorder is an immune cell induced inflammation.

7. The method claim 1, wherein the JAK-associate disorder is hepatitis.
